(19) United States
(12) Patent Application Publication
(10) Pub. No.: US 2017/0258626 A1
(43) Pub. Date: Sep. 14, 2017

(54) IMMOBILIZATION MASK

(71) Applicant: Brainlab AG, Munich (DE)

(72) Inventors: Andreas Bereket, Garching (DE); Manuel Wortmann, Feldkirchen (DE)

(21) Appl. No.: 15/504,243

(22) PCT Filed: Nov. 14, 2014
(86) PCT No.: PCT/EP2014/074577

§ 371 Date: Feb. 15, 2017

(30) Foreign Application Priority Data
Sep. 12, 2014 (EP) PCT/EP2014/069550

(51) Int. Cl. A61F 5/37

(52) U.S. Cl. CPC A61F 5/3707

(57) ABSTRACT

The present invention relates to an immobilization mask comprising a first layer comprising a low temperature thermoplastic material, the first layer having a first side and a second side; at least one dimensionally stable attachment section connected to the first layer; and at least one deformable molding member connected to the first side or to the second side of the first layer. The present invention also relates to a method of immobilizing a patient's body part, comprising the steps of applying an immobilization mask comprising a first layer having a low temperature thermoplastic material and at least one dimensionally stable attachment section connected to the first layer, wherein at least one deformable molding member is placed between the first layer and the patient's head; and molding the first layer tightly to the patient's head and to the molding member.

The present invention relates to an immobilization mask for immobilizing a body part of a patient by covering the body part's surface. Immobilization masks are used in the context of orthopedic applications, for example to immobilize joints, as well as in the context of therapeutic or diagnostic procedures, radiotherapy or diagnostic imaging, for which the body part in question has to be immobilized relative to a fixed structure and to ensure a high reproducibility of the body part's spatial position during the treatment or diagnostic procedure.

To immobilize a patient's body part, for example the patient's head, the patient's chest, a patient's shoulder or a patient's hip for radiation therapy or diagnostic imaging, the body part is connected to a fixed supporting structure such as a patient bed in a spatially fixed arrangement, and therefore in a reproducible position relative to a radiation or imaging device. For this purpose, immobilization devices are known which provide a rigid connection between the body part and the supporting structure, wherein the body part is held within a mask that has at least one sheet-like layer of a low temperature thermoplastic material, which previously has been individually adapted to the body part in a heated condition so that, after the mask has cooled down and cured, the mask tightly fits to the body part to be immobilized.

EP 1 582 187 A1 and EP 1 694 232 B1 show immobilization devices comprising a mask that is at least partly made of a thermoplastic material.

US D704338 S1 shows a head immobilization mask having a perforated sheet-like layer that fits to a patient's head.

Although known thermoplastic masks provide a tight support for immobilizing body parts, patient movement within and relative to the mask or together with the mask represents a persistent problem as any movement of the patient causes a shift of the patient position and therefore may cause an inaccurate treatment. A further problem is seen in the characteristic of many materials to absorb at least part of the radiation that is used during radiation therapy or diagnostic imaging.

It is the object of the present invention to provide an immobilization mask that overcomes at least one of the above prior art problems.

This object is solved by the subject matter of any appended independent claim. Advantages, advantageous features, advantageous embodiments and advantageous aspects of the present invention are disclosed in the following and contained in the subject-matter of the dependent claims. Different advantageous features can be combined in accordance with the invention wherever technically expedient and feasible. Specifically, a feature of one embodiment which has the same or a similar function to another feature of another embodiment can be exchanged with said other feature. A feature of one embodiment which adds an additional function to another embodiment can in particular be added to said other embodiment.

According to one embodiment, the inventive immobilization mask comprises:
a first layer comprising a low temperature thermoplastic material, the first layer having a first side and a second side;
at least one dimensionally stable attachment section connected to the first layer;
at least one deformable molding member contacting the first side or the second side of the first layer.

It is important to note that, within the scope of the present invention, different embodiments of an immobilization mask are conceivable that are configured to fit to any desirable body part, such as a patient's head, a patient's chest, a patient's shoulder, a patient's hip or any joint such as a knee joint or an elbow joint. For reasons of simplicity the following description only refers to a head immobilization mask configured to fit and to immobilize a patient's head. The inventive mask may therefore comprise one or a plurality of arbitrarily formed low temperature thermoplastic layers, depending on the specific purpose of the mask. In case the mask comprises more than one layer, the layers can have a different shape, particularly different outlines and/or differently shaped cutouts at different positions.

According to the one aspect of the present invention an immobilization mask is provided having a layer that may be delivered in an unmolded state in a planar, sheet-like form, and that comprises or is made entirely out of a low temperature thermoplastic material that will become deformable when heated to a certain temperature and will become rigid after it has cooled down again, thereby maintaining its deformed state after it has been molded to the patient's body part. Any of the two planar surfaces of the layer may be coated to meet certain requirements. For example, one or both sides may be provided with adhesive properties to bond the layer to any other structure, for example other deformable layers of the immobilization mask. On the other hand, any side of the layer may be coated with a material that prevents the layer to stick to another structure, for example to the patient's skin or hair.

The inventive immobilization mask further preferably comprises at least one dimensionally stable attachment section that is connected to the deformable layer. Since the deformable immobilization mask is part of an immobilization device, there is a need for an element that is made out of a non-low temperature thermoplastic material and maintains its shape even after the mask has been deformed so as to allow the mask to be attached to the remaining immobilization device. In a preferred embodiment the immobilization mask comprises two longitudinal attachment rails on both sides of a sheet-like layer, which may comprise through bore holes by means of which the immobilization mask can be screwed to the remaining immobilization device. However, any other detachable connections via one or more dimensionally stable attachment sections may be also conceivable.

Furthermore, the inventive immobilization mask may comprise at least one deformable molding member contacting one of the sides of the mask layer. In this context, deformable molding member means that the overall shape of the deformable molding member can be altered to conform to the contour of the patient's body part. This can be accomplished by a molding member having an elastic body, as well as by a molding member comprising a plurality of dimensionally stable elements which, like the pearls of a pearl necklace, are connected to each other so that they can change their relative position within certain limits. One can imagine that after the mask layer has been heated to a temperature that allows the layer to be deformed, the immobilization mask is applied to the head of a patient particularly to the patient's face, with the deformable molding member between the mask layer and the patient's head, the mask layer will not only adapt to the contour of the head, but also to the contour of the at least one deformable molding member lying between the mask layer and the patient's skin. By doing so, the sections of the layer which are placed above a molding member form some kind of corrugation that contributes to, in particular increases an overall rigidity of the immobilization mask.

The present invention may therefore provide a possibility to increase rigidity of a sheet-like layer of an immobilization mask, so that in the end, the risk of an inaccurate treatment caused by an unintended movement of the patient within or together with the mask is significantly reduced. On the other hand, the thickness of any mask layer with a predetermined rigidity may be reduced by the use of rigidity increasing corrugations, so that any dose build up effects and dose attenuation is minimized. The deformable molding members can take any arbitrary geometry. For example, the molding members cross-section can have a round or square form. The cross-section may also be variable over the length of the molding member. Any molding member may also have a discontinuous cross-section, i.e. may be divided into several sections.

According to a preferred embodiment of the present invention, the mask further comprises a second layer, wherein the first layer and the second layer contact each other, with the molding member lying in between the first layer and the second layer.

In contrast to an embodiment having only one deformable layer that contacts the patient's skin together with the at least one deformable molding member, an embodiment comprising two or more deformable layers of a low temperature thermoplastic material may embed the at least one deformable molding member, so that the patient's skin is in contact with the lower layer of the immobilization mask only. A mask comprising several layers allows positioning of the molding members between any pairing of the layers, thereby forming corrugations between any layer pairing. The material the second layer is made from may be different from the material of the first layer. For example, the second layer may have different deformation properties such as a lower deformation-resistance or a lower temperature threshold above which the layer becomes deformable. It is also conceivable the second layer is permanently connected to the first layer, for example adhered to the first layer or, in the alternative, only contacts the first layer, wherein a coating on the first or the second layer prevents any adhesive bond.

Any of the first and the second layer may have a perforated structure or, in the alternative, a structure without any perforation in it. Moreover, perforated sections of the first or the second layer embedded in an otherwise sleek layer are also possible.

According to a preferred embodiment of the present invention the first layer and/or the second layer comprises an inlay section having different deformation properties than the low temperature thermoplastic material of the first layer and/or the second layer.

To prevent as far as possible any movement of the patient within the mask, an inlay may be provided which is embedded within the first and/or the second layer. This inlay may take the form of a so-called “nose-bridge” provided in the vicinity of the root of the nose that will effectively prevent a nodding motion of the patient's head within the mask. With the preferred inlay provided as an integral part of a mask layer, the time necessary to mold the mask to a patient's head or face can be significantly reduced compared to a nose-bridge which is provided as an element separate to the mask layers. The material of the inlay section may have lower resistance to deformation than the low temperature thermoplastic material of the first layer and/or the second layer. This will help in adapting the form of the inlay section to the contour of a specific body part such as the root of the nose. More specifically, the inlay section may comprise a low temperature thermoplastic material different from the material of the first layer and/or the second layer. For example, the low temperature thermoplastic material of the inlay section may already deform at a lower temperature than the material of the first or the second layer. It is also conceivable that at a given temperature, the inlay material is softer than the layer material thereby helping in molding the mask to the patient's face.

Additionally or alternatively, the inlay section may have a thickness different from the thickness of the first and/or the second layer. Preferably the inlay section is thicker than the first or the second layer, or thicker than both, the first and the second layer together.

According to a further preferred embodiment of the present invention, the molding member is permanently connected to the first layer or to the second layer of the immobilization mask, wherein the layers are not connected to each other and can be handled separately. In other words, the immobilization mask is provided as an integral unit having the molding member permanently attached to one of the layers. Moreover, it is also possible to provide an “integral” immobilization mask with all of its components permanently attached to each other, i.e. the first layer is permanently connected to the at least one molding member and to the second layer. In this case, it is advantageous if the molding member is made from a radiolucent material with a low electron density, since any radiation-opaque material remaining on the treatment area would be detrimental to the treatment. As a further embodiment of the present invention, a system may be provided that comprises one or more separate mask layers with an dimensionally stable attachment section connected to at least one layer, wherein at least one molding member is provided as a further separate element, that can be applied by medical staff onto the patient's skin or onto a bottom layer before the top layer is applied and molded to the body part's and the molding member's contour.

In the alternative, the at least one molding member may be releasably connected to the first layer and/or to the second layer, such that the molding member can be removed after the mask has been molded onto a patient's head. In this case, it is not necessary that the molding member is made from radiolucent material, since it can be removed before any treatment is started. One can imagine that a removed molding member leaves a correspondingly formed hollow space under the first layer, or in case of a multi-layer-mask, between two layers, wherein the “corrugations” left in the first layer increase the overall rigidity of the mask.

As a further alternative, the at least one molding member may be just inserted between the first and the second layer without any bonding to the first or the second layer. In the case of a one-layer-mask, the at least one molding member may be connected to the first layer via a releasable bond.

Although any shape of the at least one molding member is conceivable, an elongated shape is preferred, since this will cause an elongated corrugation to be formed in the layer(s) lying above the molding member, thereby increasing the rigidity of larger areas of the mask. Moreover, the at least molding member may run across a layer over substantially the whole extent of the layer. By doing so, the corrugation generated will have the largest effect on the rigidity of the mask. A more preferred embodiment of the present invention provides a molding member running across a first layer in a direction perpendicular to the longitudinal axis of the patient's head, or in other words, from the right side of the patient's head to the left side of the patient's head.

According to a further preferred embodiment the at least molding member extends beyond the first layer's and/or the second layer's edge, which will help in removing the molding member by simply grasping and pulling the protruding end of the molding member after the mask has been molded to a patient's head and cured to a dimensionally stable state. Alternatively, at least one hole may be provided in one of the layers, through which the molding member exits the mask, preferably in the vicinity of an edge of one of the layers.

A further preferred embodiment provides a first layer and/or a second layer comprising at least one cutout for the patient's nose, mouth and/or eyes. In this respect it is noted that such cutouts must not be confused with a perforated structure of the layers, providing much smaller “holes” within the layer material.

Moreover, the at least one molding member may be stretchable to conform to the lengthening of the first layer when being molded to the patient's face. With the present invention it is recognized that molding the immobilization mask to a complex contours of the patient such as the contour of the patient's head can be performed more easily with stretchable molding members. In the alternative, non-stretchable molding member may be used. In this case, the fixed length of the at least molding member has to consider lengthening of the mask layers when being molded to the patient's head, wherein a relative motion between the molding member and the at least one mask layer is necessary.

The present invention also relates to a method of immobilizing a patient's body part comprising the following steps:
applying an immobilization mask comprising a first layer having a low temperature thermoplastic material and at least one dimensionally stable attachment section connected to the first layer, wherein at least one deformable molding member is placed between the first layer and the patient's body part;
molding the first layer tightly to the patient's body part and to the molding member.

According to one embodiment of the inventive method, any conventional sheet layer of a low temperature thermoplastic material suitable for immobilization masks may be used, wherein at least one deformable molding member is placed between the mask layer and the patient's skin together with the mask layer or before the mask layer is applied, so that after the mask layer, preferably together with the inlay, has been carefully molded to the contour of the patient's body part as well as to the contour of the at least one molding member, corrugations having a rigidity increasing effect on the overall rigidity of the mask are obtained.

The inventive method may comprise any of the technical features described in the context of the inventive immobilization mask in any expedient or feasible manner.

The present invention may be applied to any orthopedic application as well as in the technical field of radiation therapy or diagnostic imaging. In particular, the present invention may be applied to the ExacTrac® radiotherapy system, a product of Brainlab AG, Germany.

In the following, the invention is described with reference to the figures which represent preferred embodiments of the invention without limiting the invention to the specific features shown in the figures.

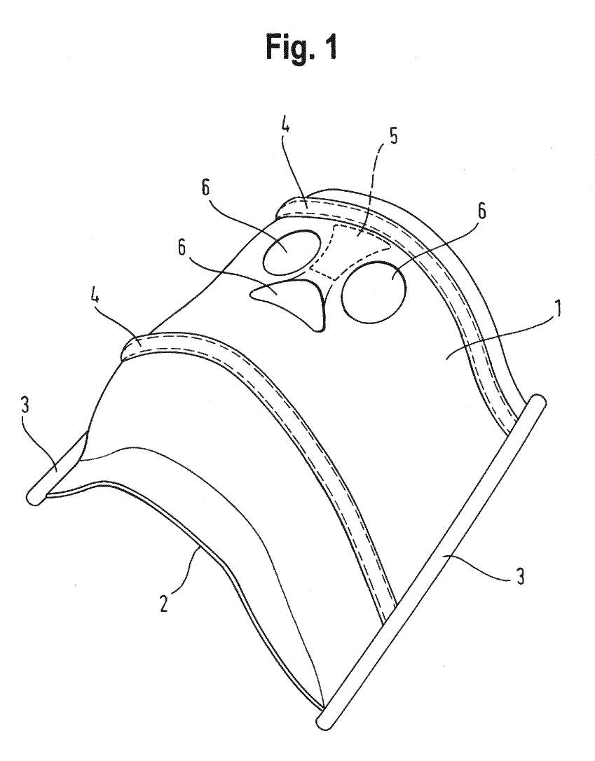

FIG. 1 shows a perspective view on a head immobilization mask being a specific embodiment of the inventive immobilization mask;

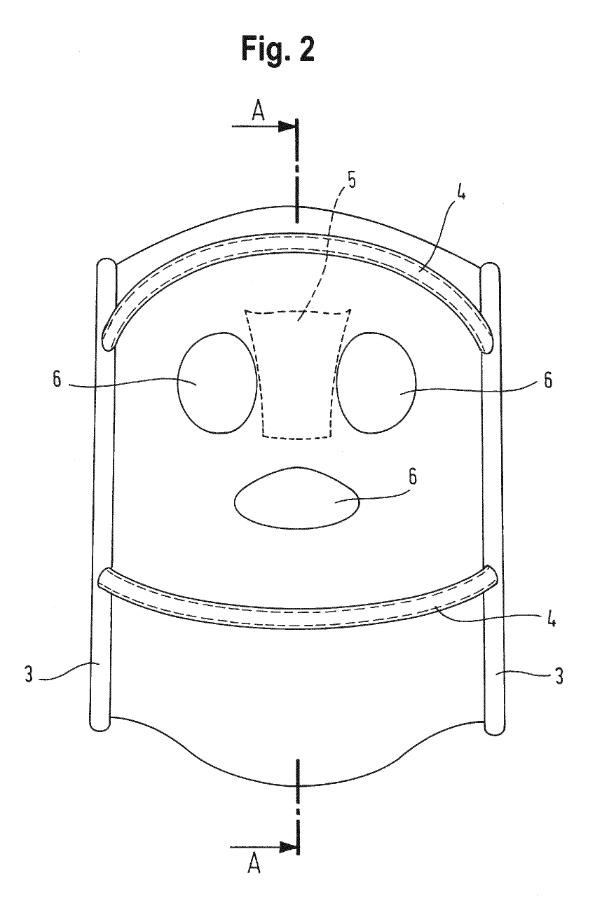

FIG. 2 shows a top view onto the mask of FIG. 1;

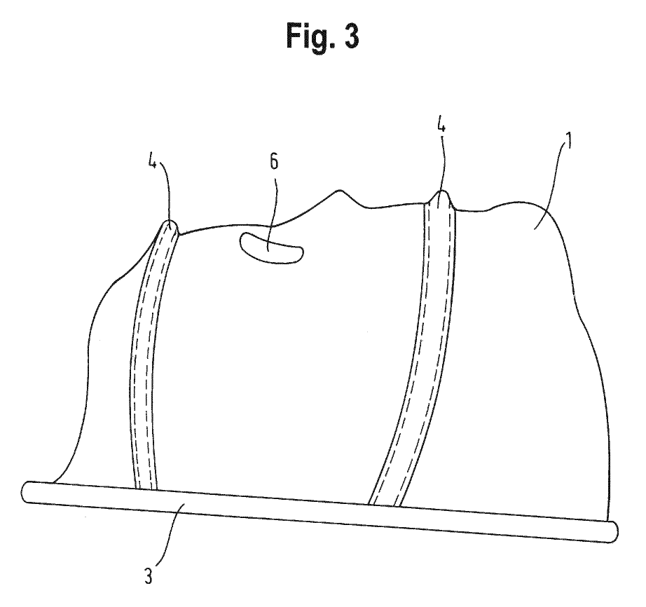

FIG. 3 shows a side view on the mask of FIG. 1;

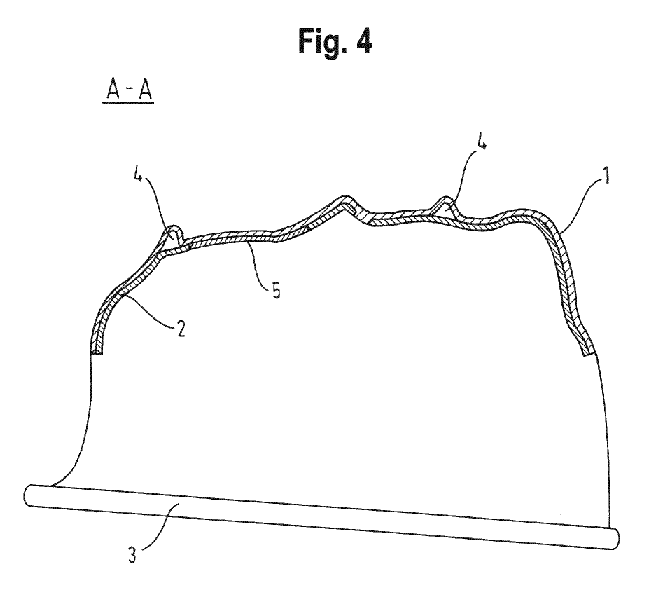

FIG. 4 shows a cross-sectional view along line A-A of the mask shown in FIG. 2;

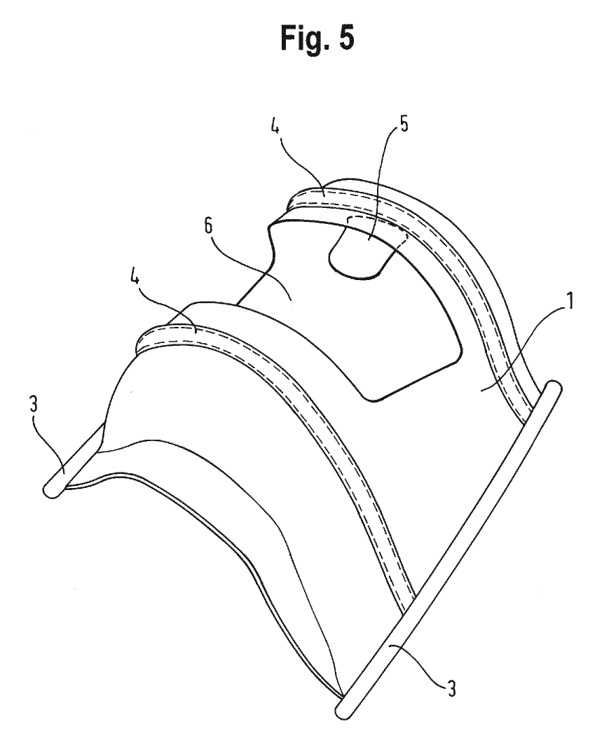

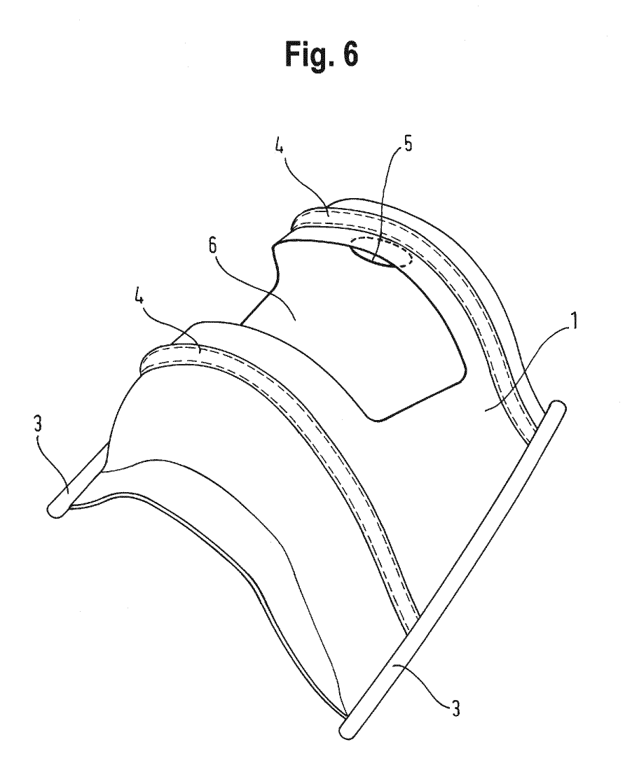

FIGS. 5 and 6 show further alternative embodiments of the present invention.

The head immobilization mask shown in FIGS. 1, 2 and 3 comprises a first upper layer 1 and a second lower layer 2 which contact each other over their whole extent and which may have substantially rectangular shape before they are molded to the patient's head. On two opposing sides of the first layer 1 and the second layer 2 two dimensionally stable attachment sections formed as rails 3 are connected to each of the layers 1 and 2, that allow the mask to be rigidly attached to a head immobilization device (not shown). Two elongated molding members 4 (shown in broken lines) are provided between the first layer 1 and the second layer 2, which cause that corresponding elongated corrugations will be formed in the first layer 1 after the mask has been molded to tightly fit to the patient's face. The corrugations run across the whole extent of the mask from the left rail 3 to the right rail 3, thereby increasing the bending resistance of the mask in a direction running alongside the corrugations. However, the inventive mask may also comprise molding members 4 running in any other suitable direction. Additionally or alternatively, the inventive mask may also comprise “interrupted” corrugations caused by interrupted elongated molding members having a discontinuous cross-section. For example, a corrugation may be formed by two molding members, wherein one molding member can be removed by pulling it to one side of the mask and the other molding member may be removed by pulling it to the other side of the mask.

The mask further comprises three cutouts 6 for the nose and both eyes of the patient, wherein in the expected vicinity of the root of the nose an insert 5 is permanently embedded in the second, lower mask layer 2, which comprises a low temperature thermoplastic material that, when being heated to a certain temperature, will become softer than the low temperature thermoplastic material of the first layer 1 and the second layer 2 to allow for molding a nose bridge to the patient's root of the nose.

FIG. 4 shows a cross-sectional view along line A-A in FIG. 2. It can be seen that after the elongated molding members 4 have been removed, hollow longitudinal tubes will be left between the upper layer 1 and the lower layer 2 that increase the mask's rigidity.

FIGS. 5 and 6 show alternative embodiments of the inventive head immobilization mask, having one single combined cutout for both, the nose and the eyes of the patient. Alternatively, the cutout can be extended to uncover the mouth and/or ear region of the patient. The embodiment shown in FIG. 5 comprises a large insert 5 that extends into the mask's cutout 6, wherein the insert 5 of the embodiment shown in FIG. 6 is mostly confined to the extent of the first and the second mask layers 1 and 2 above the combined cutout 6.

1-15. (canceled)

16. An immobilization mask, comprising:
a first layer comprising a low temperature thermoplastic material, the first layer having a first side and a second side;
at least one dimensionally stable attachment section connected to the first layer; and
at least one deformable molding member contacting the first side or the second side of the first layer.

17. The mask according to claim 16, further comprising a second layer comprising a low temperature thermoplastic material, wherein the first layer and the second layer contact each other, with the molding member lying in between the first layer and the second layer.

18. The mask according to claim 16, wherein the first layer and/or the second layer comprises an inlay section having different deformation properties than the low temperature thermoplastic material of at least one of the first layer or the second layer.

19. The mask according to claim 18, wherein the inlay section has a lower resistance to deformation than the low temperature thermoplastic material of at least one of the first layer or the second layer.

20. The mask according to claim 18, wherein the inlay section comprises a low temperature thermoplastic material different from the material of at least one of the first layer or the second layer.

21. The mask according to claim 18, wherein the material thickness of the inlay section is different from the material thickness of at least one of the first layer or the second layer.

22. The mask according to claim 16, wherein the molding member is permanently connected to at least one of the first layer or the second layer.

23. The mask according to claim 16, wherein the molding member is made from a radiolucent material.

24. The mask according to claim 16, wherein the molding member is releasably connected to the first layer or the second layer such that the molding member can be removed after the mask has been molded onto a head of a patient.

25. The mask according to claim 16, wherein the molding member has an elongated shape.

26. The mask according to claim 16, wherein at least one molding member runs across the first layer over substantially the whole extent of the first layer.

27. The mask according to claim 26, wherein at least one molding member extends beyond an edge of at least one of the first layer or the second layer.

28. The mask according to claim 16, wherein at least one of the first layer or the second layer comprises at least one cutout for at least one of a nose or eyes of a patient.

29. The mask according to claim 16, wherein the molding member is stretchable to conform to the lengthening of the first layer when being molded to a head of a patient.

30. A method of immobilizing a body part of a patient, comprising the following steps:
applying an immobilization mask comprising a first layer having a low temperature thermoplastic material and at least one dimensionally stable attachment section connected to the first layer, wherein at least one deformable molding member is placed between the first layer and the patient's body part; and
molding the first layer tightly to the patient's body part and to the molding member.

31. An immobilization mask configured to be molded to a patient's body part, comprising:
a first layer comprising a low temperature thermoplastic material, the first layer having a first side and a second side;
at least two dimensionally stable attachment sections connected to opposing ends of the first layer; and
at least one deformable molding member contacting the first side or the second side of the first layer, wherein
the first layer adapts to the contour of the patient's body part and also to the contour of the at least one deformable molding member.

32. The immobilization mask of claim 31 wherein the at least one deformable molding member is placed between the first layer and the patient's body part.

33. The immobilization mask of claim 32 wherein the at least one deformable molding member forms a corrugation that increases an overall rigidity of the immobilization mask.

34. The immobilization mask of claim 31 wherein the at least one deformable molding member is releasably connected to the first layer such that the molding member can be removed after the mask has been molded onto a head of a patient.

35. The immobilization mask of claim 31 wherein the at least one molding member is stretchable.
